(19) United States
(12) Patent Application Publication
(10) Pub. No.: US 2016/0114039 A1
(43) Pub. Date: Apr. 28, 2016

(54) TREATMENT OF POLYPOIDAL CHOROIDAL VASCULOPATHY

(71) Applicant: BAYER HEALTHCARE LLC, Whippany, NJ (US)

(72) Inventor: Oliver ZEITZ, Berlin (DE)

(21) Appl. No.: 14/893,892

(22) PCT Filed: May 20, 2014
(86) PCT No.: PCT/EP2014/060347

§ 371 Date: Nov. 24, 2015

(30) Foreign Application Priority Data
May 24, 2013 (EP) 13169079.4

(51) Int. Cl. A61K 41/00; C07K 16/28; A61K 39/395

(52) U.S. Cl. CPC A61K 41/0076; A61K 39/3955; C07K 16/2863; C07K 2319/30

(57) ABSTRACT

Methods for treatment of Polypoidal choroidal vasculopathy (PCV) of the AMD or non-AMD type and pharmaceutical compositions for the use therein are disclosed.

Polypoidal choroidal vasculopathy (PCV) is characterized by polypoidal dilations of networks of abnormal choroidal vessels. Retinal pigment epithelial (RPE) detachment, serous exudation and hemorrhages are sequelae of PCV and lead to retinal dysfunction and loss of visual acuity.

PCV was first described by Yannuzzi; 1982. Kleiner et al. (Kleiner et al. 1990) then termed it posterior uveal bleeding syndrome which subsequently became known as PCV.

PCV is particularly prevalent in Asians. Clinical studies suggest that 24.6% to 64.5% of Asian wet age-related macular degeneration (AMD) patients suffer from this subtype (Saito et al. 2008; Byeon et al. 2008; Gomi et al. 2008). In Europe, prevalence is estimated to be 4.0% to 9.8% of the wet AMD population (Ladas et al. 2004; Scassellati-Sforzolini et al. 2001; Lafaut et al. 2000). One single study in South America suggests a prevalence of 10.6% of wet AMD patients (de Mello et al. 2007).

PCV is categorized by some experts as a subtype of neovascular AMD (nAMD) or wet AMD (wAMD) but other considers it a different disease entity for the following reasons: Patients with PCV are mostly Asians or African-Americans and on the average they are younger than the patients diagnosed with AMD. Their eyes have considerably less, or even a complete lack of drusen, which are a characteristic sign of AMD (Laude et al., 2010).

The best technique currently available from differentiating PCV from other types of neovascularization like wet AMD is indocyanine green angiography (ICGA). Based on ICGA findings, PCV is defined as the presence of single or multiple focal nodular areas of hyperfluorescence arising from the choroidal circulation. (Koh et al. 2012). Fluorescein angiography (FA) is not as useful as ICGA because PCV lesions on FA resemble occult choroidal neovascularization (CNV) lesions and when submacular, they can be mistaken for wet AMD.

ICGA-guided thermal laser photocoagulation is performed for active extrafoveal polyps. Photodynamic therapy (PDT) with Visudyne® (V®-PDT) or combination of V®-PDT and anti-VEGF therapy are treatment modalities for active juxtafoveal and subfoveal lesions. If V®-PDT is contraindicated anti-VEGF monotherapy is recommended. Furthermore, anti-VEGF monotherapy is also recommended, when V®-PDT or combination of V®-PDT and anti-VEGF therapy resulted in complete regression of polyps but leakage on FA with clinical signs of activity are still detectable (Koh et al. 2013).

In the EVEREST study, which was a multi-center, double-masked trial three treatment regimens were compared: V®-PDT plus the anti-VEGF agent ranibizumab (Lucentis), ranibizumab monotherapy, and V®-PDT monotherapy. The patient population was sixty-one Asian patients with symptomatic PCV. The primary end point was complete polyp regression as assessed at month 6. V®-PDT combined with ranibizumab or alone was superior to ranibizumab monotherapy in achieving complete polyp regression (77.8% and 71.4% vs. 28.6%; Koh et al. 2012).

It is worth to note that the polyp regression observed in the EVEREST study after combination therapy is not as high as the addition of the polyp regression of the single therapies (77.8% versus 71.4%+28.6%). That indicates that a sub-population of the patient cohort responds differently to the combination therapy. Taken together that PCV and AMD have similar and differential features and that clinical trial results suggest a subpopulation which responds differently to the treatment indicates the segmentation of PCV into an “AMD type” and into a “non-AMD type” which should be treated with different treatment regimes.

The present invention is to provide specific treatment regimens for PCV of the “AMD type” and for PCV of the “non-AMD type”.

The terminology for the two types of PCV is preliminary. Alternate terms for the “AMD type” of PCV may include:
1) “AMD-like PCV”
2) “AMD with polyps”
3) “PCV with AMD”
4) “PCV with Drusen”
5) “Age-related PCV”
6) PCV Type 1
7) PCV Type 2
8) PCV Type X, where X is any number, letter or combination of both

Alternate terms for the “non-AMD type” of PCV may include:
1) “Primary PCV”
2) “PCV in young patients”
3) “PCV of the young”
4) “Central serous retinopathy-like PCV”
5) “Central serous retinopathy with PCV”
6) “PCV with central serous retinopathy”
7) “multifocal PCV”
8) “Young patient PCV”
9) “juvenile PCV”
10) “atypical AMD”
11) PCV Type 1
12) PCV Type 2
13) PCV Type X, where X is any number, letter or combination of both

In the following, the terms “AMD type” and “non-AMD type” will be used.

While the presence of polyps and thereby the diagnosis of PCV is usually confirmed by indocyanine green angiography (ICGA), the two PCV types can be differentiated as follows:
1) AMD-type: Hallmarks of AMD need to be present. These hallmarks include, but are not necessarily limited to Drusen, AMD-typical age (>=50 years) and AMD-typical RPE changes, like for example RPE hyperplasia, RPE pigment changes, or RPE atrophy.
2) Non-AMD type: No hallmarks of AMD present. The funduscopic signs may show similarities to central serous retinopathy (CSR), like for example blurry vision, fluid accumulation under the retina or leakage in the FA, which appears in many cases in a typical “smoke stack” shape and the differential diagnosis may be considered by the treating physicians.

To achieve optimal outcomes for the patient, the treatment of PCV is different for the AMD type and for the non-AMD type.
1) Treatment for the AMD type may be as follows:
   a. Intravitreal anti-VEGF monotherapy similar to the treatment of nAMD without PCV, whereas anti-VEGF therapy refers to all approved and non-approved treatments aiming to attenuate free VEGF in the eye. This includes particularly aflibercept, ranibizumab, bevacizumab and pegaptanib, but is not limited to these compounds. Anti-VEGF treatment may be applied according to the following treatment schedules:
      i. Three monthly intravitreal injections or three intravitreal injections each 4 weeks apart followed by dosing every other month or every 4 weeks with or without the option to extend the treatment interval further during the later treatment phase
      ii. Treatment until visual acuity and/or retinal morphology (e.g. as assessed by OCT, Fluoresceine Angiography, Indocyanine Angiography, Funduscopy, etc.) stabilizes, then discontinuation of treatment. Re-initiation of treatment upon deterioration of visual acuity and/or retinal morphology.
      iii. Any as needed (pro-re-nata—“PRN”) regimen
      iv. Any Treat&Extend regimen
      v. Any other treatment regimen that is or has been used for treatment of nAMD
   b. Therapy with one or more of the following treatments. If more than one treatment is used, they may be used at the same time or sequentially.
      i. Anti-VEGF treatment as described under 1)
      ii. Single or repeated applications of photodynamic therapy with Visudyne® (V®-PDT)
      iii. Single or repeated applications of steroids (all available local or systemic application routes) including slow-release or depot formulations (e.g. Ozurdex, triamcinolone, dexamethasone, Iluvien, etc.)
      iv. Radiation therapy
      v. Thermal laser therapy including sub-threshold treatments
      vi. Surgical therapy
      vii. Pharmacological vitreolysis (e.g. with Jetria or other approved or non-approved drugs)
      viii. Systemically or locally applied inhibitors of tyrosine kinases
      ix. Systemically or locally applied inhibitors of the VEGF receptor
2) Treatment for the non-AMD type may be as follows
   a. Therapy with one or more of the following treatments. If more than one treatment is used, they may be used at the same time or sequentially.
      i. Anti-VEGF treatment (including, but not limited to treatment with aflibercept, ranibizumab, pegaptanib-sodium and bevacizumab; treatment schedules as described under 1)a.)
      ii. Single or repeated applications of photodynamic therapy with Visudyne® (V®-PDT)
      iii. Single or repeated applications of steroids (all available local or systemic application routes) including slow-release or depot formulations (e.g. Ozurdex, triamcinolone, dexamethasone, Iluvien, etc.)
      iv. Radiation therapy
      v. Thermal laser therapy including sub-threshold treatments
      vi. Surgical therapy
      vii. Pharmacological vitreolysis (e.g. with Jetria or other approved or non-approved drugs)
      viii. Systemically or locally applied inhibitors of tyrosine kinases
      ix. Systemically or locally applied inhibitors of the VEGF receptor
3) Treatment schedule for combination therapy for both types of PCV: If a combination therapy is applied, the following treatment schedules may be applicable
   a. Initiate therapy with anti-VEGF therapy. Begin with a single injection or with two, three, four, five, six or more injections each one, two, three or more months or 4, 8, 12 or more weeks apart. One, two, three or more months or 4, 8, 12 or more weeks after the last injection determine whether the patient responded well. If not, add or switch to another therapy from the list under 2)a.
   b. Treat continuously or as needed with anti-VEGF as described under 1)a. If the treatment response is not satisfactory, add one or more additional therapies from the list provided under 2)a.
   c. Treat continuously or as needed with anti-VEGF as described under 1)a. If the treatment response is not satisfactory, add one or more additional therapies from the list provided under 2)a. After addition of one or more therapies change the treatment schedule for anti-VEGF, e.g. by extending the treatment interval, switching from a fixed dosing to an as needed dosing, temporarily or permanently stopping the treatment with anti-VEGF
   d. Initiate therapy with anti-VEGF therapy as described under 1)a or treat continuously or as needed with anti-VEGF as described under 1)a. If the treatment response is not satisfactory, continue treatment with a combination of anti-VEGF therapy and PDT with Visudyne® (V®-PDT) for one, two, three, four, five, six or more times each one, two, three or more months or 4, 8, 12, or more weeks apart. For the combination of anti-VEGF therapy and V®-PDT the administration of the pharmaceutical composition for anti-VEGF-therapy to the patient is followed by V®-PDT at the same day, or 1-7 days, or 2-3 days after the administration of the anti-VEGF-therapy.

While the invention covers all aforementioned therapy options, treatment of the non-AMD type of PCV with a combination therapy including single or repeated applications of V®-PDT as for example described under 3)d or including thermal laser therapy, which includes sub-threshold treatments is preferred, while for the AMD-type of PCV a monotherapy—mirroring the treatment for nAMD—is envisioned.

The invention also relates to pharmaceutical compositions or medicaments for the above mentioned treatment schemes.

### EXAMPLE 1

The aim of the study is to compare aflibercept monotherapy with aflibercept plus PDT combination therapy for the treatment of patients with PCV in a randomized, double-masked, multi-center clinical trial. Furthermore the efficacy of aflibercept monotherapy and aflibercept plus PDT combination therapy between patients with AMD-type of PCV and non-AMD-type of PCV will be compared retrospectively.

### Patient Inclusion Criteria:

Men and women with active PCV confirmed by indocyanine green angiography (ICGA) are included. The greatest linear dimension of a lesion in the study eye is <5400 mm (approximately, 9 Macular Photocoagulation Study disk areas) as assessed by ICGA. Furthermore ETDRS Best-corrected visual acuity (BCVA) assessment of the study eye results in 73 to 24 letters.

### Study Design:

The duration of the study is 96 weeks.

Week 0-8: Patients receive 3 initial intravitreal monthly doses of 2 mg aflibercept.

Week 12: Patients are randomized in two groups:
Group 1: aflibercept+sham PDT
Group 2: aflibercept+V®-PDT

Randomization will be based upon the presence or absence of qualification for rescue therapy as specified in the “Rescue therapy criteria” and by ethnicity (Japanese or non-Japanese).

Week 16-52: All patients either continue to receive aflibercept bi-monthly or receive rescue therapy (monthly aflibercept plus sham PDT (Group 1) or monthly aflibercept plus V®-PDT (Group 2)) if they meet the “Rescue therapy criteria”.

Week 53-96: Patients of both groups are treated under a treat-and-extend regimen. If patients meet the “Rescue therapy criteria” they are treated with aflibercept+sham PDT (Group 1) or with aflibercept+V®-PDT treatment (Group 2).

Week 0, 12 52, 96: BCVA, OCT, fundus photography and ICGA is performed.

### Rescue Therapy Criteria:

“Rescue therapy criteria” are evaluated at each visit. In order to assess the “Rescue therapy criteria” BCVA and/or OCT are performed. If the results indicate rescue treatment fundus photography and ICGA is performed.

All of the following three criteria must be met:
Best-corrected visual acuity of ≦73 Early Treatment Diabetic Retinopathy Study letters
Evidence of new or persistent fluid on optical coherence tomography
Evidence of active polyps on ICGA

Additionally, one of the following criteria must be fulfilled:
Deterioration, no change, or insufficient improvement in best-corrected visual acuity from baseline of <5 letters, or
Improvement in best-corrected visual acuity from baseline of ≧5 letters, but <10 letters, and the investigator determines based on the course of visual and anatomic outcomes over time that photodynamic therapy might be of additional benefit to the subject.

### Aflibercept Treatment:

2 mg (0.05 mL) aflibercept is administered by intravitreal injection.

### V-PDT Treatment:

For patients who meet the need for rescue therapy and who were assigned to Group 2, 6 mg/m² verteporfin (Visudyne®) will be given intravenously according to the label. Treatment with photodynamic therapy may be delivered on the same visit day as aflibercept, preferably after the administration of aflibercept. If the administration of PDT is delayed, it should be administered within 2-3 days after ICGA/FA, but not later than 7 days. PDT may only be administered if at least 3 months have elapsed since the last PDT administration (per label use).

### Best-Corrected Visual Acuity (BCVA)

Visual function of the study eye and the fellow eye is assessed at each study visit according to the standard procedures developed for the ETDRS adapted for Age Related Eye Disease Study (AREDS).

### Optical Coherence Tomography (OCT)

Retinal and lesion characteristics, such as central retinal thickness (CRT), are evaluated by OCT in both eyes at every study visit.

### Fundus Photography (FP)/Fluorescein Angiography (FA)/Indocyanine Green Angiography (ICGA)

The anatomical state of the retinal vasculature of the study eye (e.g. CNV lesion size) is evaluated by funduscopic examination, FP and FA, including ICGA. Fundus photography and FA are obtained at the screening visit, Week 12, Week 52, and Week 96 visits and in addition if deemed necessary by patient status. Additional examinations may be performed when the results from BCVA indicates that the subject may qualify for rescue therapy.

### REFERENCES

Byeon S H, Lee S C, Oh H S, Kim S S, Koh H J, Kwon O W.
Incidence and clinical patterns of polypoidal choroidal vasculopathy in Koreanpatients.
Jpn J Ophthalmol.2008 January-February;52(1):57-62. Epub 2008 Mar. 28.
Gomi F, Ohji M, Sayanagi K, Sawa M, Sakaguchi H, Oshima Y, Ikuno Y, Tano Y.
One-year outcomes of photodynamic therapy in age-related macular degeneration and polypoidal choroidal vasculopathy in Japanese patients.
Ophthalmology.2008 January;115(1):141-6. Epub 2007 Jun. 20.
Kleiner R C, Brucker A J, Johnston R L.
The posterior uveal bleeding syndrome.
Retina 1990; 10:9-17.
Koh A H, Lee W K, Chen L J, Chen S J, Hashad Y, Kim H, Lai T Y, Pilz S, Ruamviboonsuk P, Tokaji E, Weisberger A, Lim T H.
EVEREST STUDY: Efficacy and Safety of Verteporfin Photodynamic Therapy in combination with Ranibizumab or Alone Versus Ranibizumab Monotherapy in Patients with Symptomatic Macular Polypoidal Choroidal Vasculopathy.
Retina 2012; 32:1453-1464.
Koh A H, Chen L J, Chen S J, Chen Y, Giridhar A, lida T, Kim H, Yuk Yau Lai T, Lee W K, Li X, Han Lim T, Ruamviboonsuk P, Sharma T, Tang S, Yuzawa M
Polypoidal choroidal vasculopathy: evidence-based guidelines for clinical diagnosis and treatment.
Retina. 2013 33(4):686-716
Ladas I D, Rouvas A A, Moschos M M, Synodinos E E, Karagiannis D A, Koutsandrea C N

Polypoidal choroidal vasculopathy and exudative age-related macular degeneration in Greek population.
Eye.2004 May;18(5):455-9.
Lafaut B A, Leys A M, Snyers B, et al.
Polypoidal choroidal vasculopathy in Caucasians.
Graefes Arch Clin Exp Ophthalmol. 2000; 238:752-759
Laude, A., Cackett, P. D., Vithana, E. N., Yeo, I. Y., Wong, D., Koh, A. H., Wong, T. Y., Aung, T.,.
Polypoidal choroidal vasculopathy and neovascular age-related macular degeneration: same or different disease?
Prog.Retin.EyeRes. 2010; 29,19-29.
de Mello P C, Brasil O F, Maia H S, Kaiser P K, Pereira M B, de Moraes H V Jr.
Prevalence and epidemiologic features of polypoidal choroidal vasculopathy in southeastern Brazil.
Eye.2007 September;21(9):1247.
Saito M, lida T, Nagayama D.
Photodynamic therapy with verteporfin for age-related macular degeneration or polypoidal choroidal vasculopathy: comparison of the presence of serous retinal pigment epithelial detachment.
Br J Ophthalmol.2008 December;92(12):1642-7
Scassellati-Sforzolini B, Mariotti C, Bryan R, et al.
Polypoidal choroidal vasculopathy in Italy.
Retina. 2001;21:121-125.
Yannuzzi L A.
Idiopathic polypoidalchoroidal vasculopathy.
Macula Society Meeting, 1982, Miami, Fla.

1. A method for treating Polypoidal Choroidal Vasculopathy (PCV) in a patient, comprising establishing if the PCV is of AMD-type or non-AMD type, and then treating the patient according to usual AMD treatment schemes in case of AMD type PCV.

2. The method according to claim 1, wherein in case of a non-AMD type PCV, the patient is treated with a combination therapy including single or repeated applications of photodynamic therapy with Visudyne® (V®-PDT).

3. The method according to claim 2, wherein the combination therapy includes anti VEGF treatment.

4. The method according to claim 3, wherein the anti-VEGF treatment includes administration of a compound selected from aflibercept, ranibizumab, bevacizumap, or pegaptanip.

5. The method according to claim 1, wherein in case of a AMD-type PCV the treatment is a monotherapy.

6. The method according to claim 1, wherein the treatment is an anti-VEGF therapy.

7. The method according to claim 6, wherein the anti-VEGF therapy includes administration of a compounds selected from aflibercept, ranibizumab, bevacizumap, or pegaptanip.

8. A pharmaceutical composition comprising Visudyne for PDT-treatment of non-AMD type PCV.

9. The a pharmaceutical composition according to claim 8, comprising Visudyne® for PDT-treatment of non-AMD type PCV in a combination therapy.

10. The a pharmaceutical composition according to claim 8, comprising Visudyne® for PDT-treatment of non-AMD type PCV in an anti-VEGF combination therapy.

11. The a pharmaceutical composition according to claim 10, comprising Visudyne® for PDT-treatment of non-AMD type PCV in a combination therapy comprising aflibercept, ranibizumab, bevacizumap, or pegaptanip.

12. A pharmaceutical composition comprising aflibercept, ranibizumab, bevacizumap, or pegaptanip for therapy of non-AMD type PCV.

13. The pharmaceutical composition of claim 12 comprising aflibercept for therapy of a non-AMD type PCV.

14. A pharmaceutical composition for the treatment of PCV comprising an anti-VEGF agent wherein it is first established if the PCV is of AMD type or non-AMD type and then in case of AMD-type an AMD treatment is selected.

15. A pharmaceutical composition for the treatment of PCV comprising an anti-VEGF agent wherein it is first established if the PCV is of AMD type or non-AMD type and then in case of non-AMD type PCV the patient is treated with a combination therapy including single or repeated applications of PDT with Visudyne®.

16. The a pharmaceutical composition according to claim 15, wherein in case of AMD-type PCV a monotherapy is applied and in case of non-AMD type PCV a combination therapy is applied.

17. The pharmaceutical composition according to claim 16, wherein in case of non-AMD type PCV the anti-VEGF therapy is combined with comprising Visudyne® treatment.

18. The pharmaceutical composition according to claim 15, wherein the anti-VEGF agent is selected from the group consisting of aflibercept, ranibizumab, bevacizumap, or pegaptanip.

19. A method for the treatment of PCV in a patient, comprising
a. administering an initial anti-VEGF-therapy to the patient
b. a subsequently evaluating response to the initial anti-VEGF-therapy; and
c. administering a subsequent single or repeated combination treatment comprising anti-VEGF-therapy in the case the patient does not responds to the initial anti-VEGF therapy.

20. The method according to claim 19, wherein the combination treatment comprises PDT and Visudyne® wherein:
a. administering the pharmaceutical composition for anti-VEGF-therapy to the patient and,
b. subsequently a PDT treatment with Visudyne® is performed on the same day as the anti-VEGF-therapy, or 1-7, or 2-3 days after anti-VEGF-therapy.

21. The method according to claim 19, wherein the initial anti-VEGF-therapy comprises of a single injection or two, three, four, five, six or more injections of the pharmaceutical composition for anti-VEGF therapy each 4, 8, 12 or more weeks apart.

22. The method according to claim 19, wherein at least up to 3 doses of the anti-VEGF-therapy are administered every 4 weeks.

23. The method according to claim 19, wherein the evaluation of the treatment response is performed 4, 8, 12 or more weeks after the preceding anti-VEGF-therapy.

24. The method according to claim 19 wherein the secondary combination therapy is performed one, two, three, four, five, six or more times each 4, 8, 12 or more weeks apart.

25. The method according to claim 19, wherein the anti-VEGF-therapy includes administration of a compound selected from aflibercept, ranibizumab, bevacizumap, or pegaptanip.

26. The method according to claim 1, wherein in case of non-AMD type PCV the patient is treated with a combination therapy including thermal laser therapy, which includes sub-threshold treatments.

27. A pharmaceutical composition for the treatment of PCV comprising an anti-VEGF agent wherein it is first established if the PCV is of AMD type or non-AMD type and then in case of non AMD-type PCV the patient is treated with a combination therapy including thermal laser therapy, which includes sub-threshold treatments.
